(19) United States
(12) Patent Application Publication
(10) Pub. No.: US 2009/0155182 A1
(43) Pub. Date: Jun. 18, 2009

(54) OPTICAL IN VIVO IMAGING CONTRAST AGENTS AND METHODS OF USE

(75) Inventors: John Mauro, Eugene, OR (US); Julie Nyhus, Eugene, OR (US); Thomas Steinberg, Eugene, OR (US); Yu-Zhong Zhang, Eugene, OR (US)

Correspondence Address: INVITROGEN CORPORATION;C/O INTELLEVATE, P.O. BOX 52050, MINNEAPOLIS, MN, 55402, US

(73) Assignee: Invitrogen Corporation, Carlsbad, CA (US)

(21) Appl. No.: 12/096,790

(22) PCT Filed: Dec. 8, 2006
(86) PCT No.: PCT/US06/61792

§ 371 Date: Oct. 20, 2008

Related U.S. Application Data
(60) Provisional application No. 60/749,211, filed on Dec. 9, 2005.
(60) Provisional application No. 60/804,928, filed on Jun. 15, 2006.

(51) Int. Cl. A61K 49/00

(52) U.S. Cl. 424/9.6

(57) ABSTRACT

Provided is an optical in vivo contrast agent comprising a fluorescent polymeric microsphere, wherein the microsphere is impregnated with a dye having an excitation and emission spectrum compatible with in vivo imaging, and wherein the microsphere is coated with a block copolymer.

### CROSS REFERENCE TO RELATED APPLICATIONS

This application claims priority to U.S. Provisional Patent Application No. 60/749,211, filed Dec. 9, 2005 and U.S. Provisional Patent Application No. 60/804,928, filed Jun. 15, 2006, both of which are hereby incorporated by reference as if set forth fully herein.

### FIELD OF THE INVENTION

The present invention relates to in vivo imaging of physiological disease states in living beings using fluorescent microspheres coated with a block copolymer. The invention has applications in the fields of cell biology, in vivo imaging, pathology, neurology, immunology, proteomics and biosensing.

### BACKGROUND OF THE INVENTION

The utility of in vivo contrast agents depends on their preferential accumulation in target tissues and attaining sufficient signal-to-noise ratios to yield satisfactory image resolution.

The use of magnetic resonance imaging contrast enhancement agents or radioactive isotopes in the body is practiced by a variety of methods. U.S. Pat. No. 5,135,737 teaches magnetic resonance imaging enhancement agents of paramagnetic metal ion chelates attached to polymers such as polyamine based molecules with antibodies attached for concentration at desired sites in the body. U.S. Pat. Nos. 4,938,947 and 5,017,359 teach an aerosol composition containing soluble fragments of bacterial wall or cell peptidoglycan which may be labeled with a paramagnetic element and encapsulated in liposomes which may be administered as an aerosol. U.S. Pat. No. 5,078,986 teaches magnetic resonance imaging agents of a chelate of a paramagnetic element carried by or within the external surface of a liposome and released at a desired organ or tissue site. PCT Publication Number WO 92/21017 teaches specific liposomes complexed with paramagnetic ions to prolong their blood pool half life and control magnetic resonance relaxivity. Liposomes as MR contrast agents has been reviewed by Unger, E. C., Shen, D. K., and Fritz, T. A., Status of Liposomes as MR Contrast Agents, JMRI, 3, 195-198, (1993).

There is considerable interest in the use of fluorescent dyes as contrast agents to differentiate diseased from normal tissue (Mahmood, 2004; M. Rudin and R. Weissleder, 2003; Grimm 2003). Although molecular organic dyes that emit in the near infrared have been developed to facilitate fluorescent imaging of diseased areas deep in the body, issues such as low tissue uptake, rapid blood clearance, uncontrolled diffusion properties and photobleaching are among the significant problems that can compromise their in vivo utility.

Several dyes that absorb and emit light in the visible and near-infrared region of electromagnetic spectrum are currently being used for various biomedical applications due to their biocompatibility, high molar absorptivity, and/or high fluorescence quantum yields. The high sensitivity of the optical modality in conjunction with dyes as contrast agents parallels that of nuclear medicine, and permits visualization of organs and tissues without the undesirable effect of ionizing radiation. The most widely used dye is cyanine dyes because they have an intense absorption and emission in the near-infrared (NIR) region and are particularly useful because biological tissues are optically transparent in this region (B. C. Wilson, Optical properties of tissues. Encyclopedia of Human Biology, 1991, 5, 587-597).

A major drawback in the use of cyanine dye derivatives is the potential for hepatobiliary toxicity resulting from the rapid clearance of these dyes by the liver (G. R. Cherrick, et al., Indocyanine green: Observations on its physical properties, plasma decay, and hepatic extraction. J. Clinical Investigation, 1960, 39, 592-600). This is associated with the tendency of cyanine dyes in solution to form aggregates, which could be taken up by Kupffer cells in the liver.

Another major difficulty with current cyanine and indocyanine dye systems is that they offer a limited scope in the ability to induce large changes in the absorption and emission properties of these dyes. Attempts have been made to incorporate various heteroatoms and cyclic moieties into the polyene chain of these dyes (L. Strekowski, et al., Substitution reactions of a nucleofugal group in hetamethine cyanine dyes. J. Org. Chem., 1992, 57, 4578-4580; N. Narayanan, and G. Patonay, A new method for the synthesis of heptamethine cyanine dyes: Synthesis of new near infrared fluorescent labels. J. Org. Chem., 1995, 60, 2391-2395; U.S. Pat. Nos. 5,732,104; 5,672,333; and 5,709,845), but the resulting dye systems do not show large differences in absorption and emission maxima, especially beyond 830 nm where photoacoustic diagnostic applications are very sensitive. They also possess a prominent hydrophobic core, which enhances liver uptake.

For the purpose of tumor detection, many conventional dyes are not useful for in vivo applications because of their highly toxic effect on both normal and abnormal tissues. Other dyes lack specificity for particular organs or tissues and, hence, these dyes must be attached to bioactive carriers such as proteins, peptides, carbohydrates, and the like to deliver the dyes to specific regions in the body.

Long-circulating particles, such as liposomes, dendrimers, semiconductor nanocrystals (quantum dots), and polymer-based micro- and nano-spheres afford opportunities to overcome some of these problems. A substantial body of theoretical and practical work has been done involving the utility and behavior of particles in living subjects (Moghimi et al, 2001).

General modes for in vivo distribution of contrast agents are active or passive with respect to targeting within tissues. Unique structural changes associated with a given vascular physiology can result in escape of particles from the circulation at sites where the capillaries have open fenestrations, as in the sinus endothelium of the liver, or when the integrity of the endothelial barrier is perturbed by inflammatory processes such as in rheumatoid arthritis, coronary infarction, certain infections, or in abnormal vascular states associated with tumors. There is evidence to support facile extravasation (movement from the blood vessel lumen into the surrounding tissue) of particles in the size range of 50-200 nm during inflammation and in certain cancers, based on experimental observations in animals and humans (Boerman et al., 1997; Dams et al., 1998, 1999, 2000; Hobbs et al, 1998). In some cases, openings in the normally tight endothelial cell barrier can be much larger, up to 2-3 microns. Such openings between defective endothelial cells can explain tumor blood vessel leakiness, and transcellular holes (holes passing through individual cells) up to 0.6 microns in diameter have been observed as well (Hashizume et al., 2000). Evidence for increased vascular permeability has come from many sources. Extravasation of soluble tracers such as radioisotopes, albumin, fibrinogen, dextran, horseradish peroxidase, lissamine green, indocyanine green, stealth liposomes, quantum dots, and ferritin has been observed in experimental tumors. The mechanism of vessel leakiness in inflammation is relatively well characterized; most studies point to the involvement of transiently-open intercellular gaps in leakage from inflamed vessels, and under some conditions transcellular holes may also play a role.

In some imaging applications, overly rapid extravasation of a contrast agent may be undesirable. For example, rapid entry into the interstitium of a tumor may obscure the boundary of the blood vessel wall. A common way to highlight blood vessels is to highlight them with fluorescently labeled dextran of molecular weight of ˜2,000,000. In McalV tumors, however, even this large molecule is rapidly extravasated, leading to boundary obscuration in images. Numerous reports exist demonstrating that long flexible molecules (e.g. dextrans) experience reduced hindrance to interstitial transport relative to more rigid and spherical particulates, use of which as contrast agents can lead to a sharper boundary between intra-and extravascular spaces (Stroh et al, 2005).

Numerous problems still exist for the use of contrast imaging agents, either in their ability to target to a specific location, imaging capabilities, or their toxic effect on the body. Thus we have developed a novel method for passive targeting, or preferential accumulation, of the contrast agent in target tissues that exist in various physiological and/or pathological states.

New and/or better contrast agents for optical in vivo imaging are needed. The present invention is towards this important end.

### SUMMARY OF THE INVENTION

In one embodiment is provided a contrast agent comprising a fluorescent microsphere, wherein the microsphere is labeled or impregnated with a dye having an excitation and emission spectrum compatible with in vivo imaging, wherein the microsphere is coated with a block copolymer. The microspheres are polymeric and in one aspect are comprised of polystyrene. The fluorescent dyes or fluorophores typically have an excitation wavelength of at least about 580 nm.

Another embodiment of the invention provides a method for imaging a site of disease or injury within the vasculature of a subject wherein the method comprises:
administering to the subject a contrast agent comprising fluorescent microspheres impregnated or labeled with a dye having an excitation and emission spectrum compatible with in vivo imaging and wherein the microspheres are coated with a surfactant;
illuminating the contrast agents in the subject with an appropriate wavelength to form an illuminated subject; and
observing the illuminated subject whereby the site of disease or injury is imaged.

In another more particular embodiment the surfactant is a block copolymer. More particularly, the block copolymer is comprised of polyoxyethylene (PEO) and polyoxypropylene (PPO) subunits. More particular still, the block copolymer is poloxamer 407.

In another more particular embodiment the microspheres comprise polystyrene.

In another more particular embodiment the dye has an excitation wavelength between about 580 nm to about 800 nm.

In another more particular embodiment the disease is arthritis, a coronary infarction, an infection, or cancer.

In another more particular embodiment the dye is selected from the group consisting of a pyrene, an anthracene, a naphthalene, an acridine, a stilbene, an indole, an oxazole, benzoxazole, a thiazole, a benzothiazole, a 4-amino-7-nitrobenz-2-oxa-1,3-diazole (NBD), a carbocyanine, a carbostyryl, a porphyrin, a salicylate, an anthranilate, an azulene, a perylene, a pyridine, a quinoline, a xanthene, an oxazine, a benzoxazine, a carbazine, a phenalenone, a coumarin, a benzofuran, a benzphenalenone, a semiconductor nanocrystal, and a fluorescent protein.

In another more particular embodiment the contrast agent is administered intravenously to the subject.

In another more particular embodiment the microspheres are coated with the surfactant in vivo. More particular still, the surfactant is a block copolymer. In another more particular embodiment the contrast agent remains at the disease or injury site for at least one, two, three, four, five, six, seven, eight, nine, ten, eleven, twelve, thirteen, fourteen, fifteen, sixteen, seventeen, eighteen, nineteen, twenty, or twenty-four hours or 1-30 days. In another embodiment the contrast agent has a very high t_{1/2}, or a very low clearance from the site of injury or disease. The contrast agents of the present invention have been observed to remain at the site of injury for extended periods of time, allowing observation of those sites to occur with out repeated dosing of the subject. Additionally, this allows for disease states, such as tumor growth or macrophage build up, to be monitored for extended periods of time, including, days, weeks, or months.

Another more particular embodiment of the invention further comprises the step of incubating said subject for a sufficient amount of time for the contrast agents to circulate to the disease or injury sites prior to illuminating the subject.

In another more particular embodiment of the invention the contrast agent is concentrated around the site of disease or injury.

Another embodiment of the invention provides a contrast agent for in-vivo imaging of disease or injury sites in a subject comprising a fluorescent microsphere, wherein the microsphere is impregnated or labeled with a dye having an excitation and emission spectrum compatible with in vivo imaging and wherein the microspheres are coated with a surfactant.

In another more particular embodiment the surfactant is a block copolymer. More particularly, the block copolymer is comprised of polyoxyethylene (PEO) and polyoxypropylene (PPO) subunits. More particular still, the block copolymer is poloxamer 407.

In another more particular embodiment the microspheres comprise polystyrene.

In another more particular embodiment the dye has an excitation wavelength between about 580 nm to about 800 nm.

In another more particular embodiment the subject is suffering from a disease selected from arthritis, a coronary infarction, an infection, or cancer.

In another more particular embodiment the dye is selected from the group consisting of a pyrene, an anthracene, a naphthalene, an acridine, a stilbene, an indole, an oxazole, benzoxazole, a thiazole, a benzothiazole, a 4-amino-7-nitrobenz-2-oxa-1,3-diazole (NBD), a carbocyanine, a carbostyryl, a porphyrin, a salicylate, an anthranilate, an azulene, a perylene, a pyridine, a quinoline, a xanthene, an oxazine, a benzoxazine, a carbazine, a phenalenone, a coumarin, a benzofuran, a benzphenalenone, a semiconductor nanocrystal, and a fluorescent protein.

Another embodiment of the invention provides a kit for in-vivo imaging of disease or injury sites in a subject comprising:
a contrast agent comprising a fluorescent microsphere, wherein the microsphere is impregnated or labeled with a dye having an excitation and emission spectrum compatible with in vivo imaging and wherein the microspheres are coated with a surfactant;
packaging; and
instructions for imaging the disease or injury site with the contrast agent.

In another more particular embodiment the surfactant is a block copolymer. More particularly, the block copolymer is comprised of polyoxyethylene (PEO) and polyoxypropylene (PPO) subunits. More particular still, the block copolymer is poloxamer 407.

In another more particular embodiment the microspheres comprise polystyrene.

In another more particular embodiment the dye has an excitation wavelength between about 580 nm to about 800 nm.

In another more particular embodiment the subject is suffering from a disease selected from arthritis, a coronary infarction, an infection, or cancer.

In another more particular embodiment the dye is selected from the group consisting of a pyrene, an anthracene, a naphthalene, an acridine, a stilbene, an indole, an oxazole, benzoxazole, a thiazole, a benzothiazole, a 4-amino-7-nitrobenz-2-oxa-1,3-diazole (NBD), a carbocyanine, a carbostyryl, a porphyrin, a salicylate, an anthranilate, an azulene, a perylene, a pyridine, a quinoline, a xanthene, an oxazine, a benzoxazine, a carbazine, a phenalenone, a coumarin, a benzofuran, a benzphenalenone, a semiconductor nanocrystal, and a fluorescent protein.

In another more particular embodiment the contrast agent is for intravenous administration to the subject.

In another more particular embodiment the contrast agent is suspended in deionized water.

Another embodiment of the invention provides an in vivo formulation comprising any of the compositions described herein. In particular the formulation comprises fluorescent microspheres impregnated or labeled with a dye having an excitation and emission spectrum compatible with in vivo imaging and wherein the microspheres are coated with a surfactant. More particularly, the formulation comprises deionized water. More particular the formulation is sterilized.

Another embodiment of the invention provides a composition comprising blood, macrophages (or another particle associated with disease or injury to the vasculature, including leukocytes, platelets, or inflammatory particles such as adhesion molecules) and a contrast agent comprising fluorescent microspheres impregnated or labeled with a dye having an excitation and emission spectrum compatible with in vivo imaging and wherein the microspheres are coated with a surfactant.

In another embodiment is provided a method for using the present contrast agents wherein they are injected into a body and then subsequently imaged and visualized after a sufficient amount of time.

Further embodiments of the invention include those provided in the detailed description.

### BRIEF DESCRIPTION OF THE FIGURES

FIG. 1: Shows a representation of a normal and tumor blood vessel with the present contrast agents.

FIG. 2: Shows a close up image of inflamed areas of rear paw of live balbc mouse with experimentally induced arthritis 24 hours after injection with Contrast Agent 1.

FIG. 3: is two example of block copolymer used to coat the present fluorescent microspheres.

FIG. 4: Shows the effect of microspheres coated with and without Pluronic F-127. The microspheres without Pluronic F-127 pooled in the liver 24 hours after injection; the microspheres with Pluronic F-127 did not sequester in the liver, making them a more effective contrast agent.

FIG. 5: Shows an image of the accumulation of the present contrast agent around a mouse ear punch.

FIG. 6: Shows an image of the accumulation of contrast agent 1 at the site of inflammation in three of the paws of a mouse, the four was not inflamed and demonstrated no visible contrast agent 1 at the site.

FIG. 7: Shows a time course of images using contrast agent 1 from a single inflamed paw.

FIG. 8: Shows the qualitative date of the same time course from FIG. 7.

FIG. 9: Shows an image of accumulation of contrast agent 2 at the heel joint of a mouse with experimentally induced arthritis.

FIG. 10: Shows a time course of images using contrast agent 2 from a single inflamed paw.

### DETAILED DESCRIPTION OF THE INVENTION

### Introduction

The present invention provides a novel contrast agent formulation for in vivo imaging. These contrast agents are polymeric microspheres (also herein referred to as microparticles) that have been stained with a fluorescent dye(s) having an excitation wavelength compatible with in vivo imaging, typically about 580 nm to about 800 nm, and that have been coated with a block copolymer (also herein referred to as a surfactant). The coated microspheres travel relatively freely within the circulating blood until their preferential sequestration occurs at a diseased or injury tissue sites.

The present contrast agents and their use for in vivo imaging have many advantages compared to known contrast agents, and in a preferred embodiment these advantages, include, but are not limited to:
Polystyrene microspheres in the size range (100 to 2000 nm diameter) have no known intrinsic toxicity and are likely to be non-immunogenic.
Each particle contains many dye molecules and emits light intensely
The impregnated dye is protected within the polymer sphere from chemical and photodegradation.
A wide range of sizes, surface treatments and emission wavelengths can be easily prepared.
Emulsion polymerization results in highly uniform spheres and a high degree of structural and functional homogeneity.
The in vivo images produced demonstrate a high degree of localization within diseased vasculature (higher resolution than other commercially available contrast agents based on dye-labeled macromolecules).
The inert nature of the dye impregnated microspheres leads to long in vivo residence times and thus long imaging times when entrapped in tissue.
Block copolymer coating can be employed to minimize organ sequestration in non-invasive imaging applications.
Vascular contrast agents in a wide range of colors and sizes can be prepared using relatively well-known materials and processing methods.
Very bright images with much higher intrinsic contrast are possible with the present formulation than have been previously observed with available optical contrast agents.
Unique applications such as monitoring or grading vascular leakiness in disease states can be envisioned using these uniquely uniform particles.
These materials could be acceptable for both animal and human imaging due to their low toxicity.

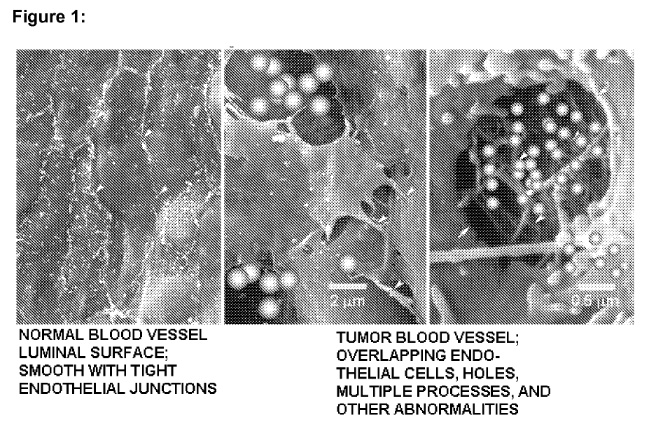

Thus, the present fluorescent microspheres function as contrast agents, optimally coated with a block copolymer, such that after injection into a subject (for instance an animal with experimentally induced disease, such arthritis or cancer) the present microspheres migrate to sites within the body distant from the injection point and accumulate in tissues in which excessive or otherwise abnormal blood vessel development occurs as part of the disease process (See FIG. 1; microspheres drawn approximately to scale—middle panel ˜1000 nm/left panel ˜100 nm).

### Definitions

Before describing the present invention in detail, it is to be understood that this invention is not limited to specific compositions or process steps, as such may vary. It must be noted that, as used in this specification and the appended claims, the singular form “a”, “an” and “the” include plural referents unless the context clearly dictates otherwise.

Unless defined otherwise, all technical and scientific terms used herein have the same meaning as commonly understood by one of ordinary skill in the art to which this invention is related. The following terms are defined for purposes of the invention as described herein.

The term “aqueous solution” as used herein refers to a solution that is predominantly water and retains the solution characteristics of water. Where the aqueous solution contains solvents in addition to water, water is typically the predominant solvent.

The term “contrast agent” as used herein refers to a plurality of fluorescent microspheres, wherein the microsphere are impregnated or labeled with a dye and coated with a surfactant. The contrast agents of the present invention have a particular ability to concentrate at disease or injury sites. Being “concentrated” at a disease or injury site, indicates a detectable number of microspheres are localized around a particular site(s). Preferably, the image obtained from illuminating and detecting the microspheres will provide specific pathological information for diagnosis and treatment options.

The term “detectable response” as used herein refers to a change in or an occurrence of, a signal that is directly or indirectly detectable either by observation or by instrumentation and the presence or magnitude of which is a function of the presence of a target in the test sample. Typically, the detectable response is an optical response resulting in a change in the wavelength distribution patterns or intensity of absorbance or fluorescence or a change in light scatter, fluorescence quantum yield, fluorescence lifetime, fluorescence polarization, a shift in excitation or emission wavelength or a combination of the above parameters. The detectable change in a given spectral property is generally an increase or a decrease. However, spectral changes that result in an enhancement of fluorescence intensity and/or a shift in the wavelength of fluorescence emission or excitation are also useful.

The term “fluorophore” as used herein refers to a composition that is inherently fluorescent. Fluorophores may be substituted to alter the solubility, spectral properties or physical properties of the fluorophore. Numerous fluorophores are known to those skilled in the art and include, but are not limited to coumarin, acridine, furan, dansyl, cyanine, pyrene, naphthalene, benzofurans, quinolines, quinazolinones, indoles, benzazoles, borapolyazaindacenes, oxazine and xanthenes, with the latter including fluoresceins, rhodamines, rosamine and rhodols as well as other fluorophores described in RICHARD P. HAUGLAND, MOLECULAR PROBES HANDBOOK OF FLUORESCENT PROBES AND RESEARCH CHEMICALS (9^{th} edition, including the CD-ROM, September 2002). As used herein fluorophores of the present invention are compatible with in vivo imaging, optically excited in tissue, and generally have an excitation wavelength of about 580 nm to about 800 nm or longer.

The term “fluorescent microsphere” as used herein refers to approximately spherical particles of size ranging from about 0.01 to 50 microns with intimately associated fluorescent material such as an organic dye, inorganic nanocrystal or metal complex

The term “illuminating” as used herein refers to the application of any light source, including near-infrared (NIR) and visible light, capable of exciting dyes impregnated within the microspheres of the invention.

The term “in vivo imaging” as used herein refers to methods or processes in which the structural, functional, or physiological state of a living being is examinable without the need for life ending sacrifice.

The term “non invasive in vivo imaging” as used herein refers to methods or processes in which the structural, functional, or physiological state of a being is examinable by remote physical probing without the need for breaching the physical integrity of the outer (skin) or inner (accessible orifices) surfaces of the body.

The term “kit” as used refers to a packaged set of related components, typically one or more compounds or compositions.

The term “microsphere or microparticle” as used herein refers to particles of a size typically measured in the range from about 0.01 to about 10 microns and composed of any organic or inorganic material whose chemical and physical properties allow formation of functionally stable particles in this size range.

The term “polymeric microsphere” as used herein refers to particles of a size typically measured in the range from about 0.01 to about 10 microns synthesized by means of chemically-catalyzed addition of monomeric molecules to chemical chains and controlled in such a way as to achieve particles of uniform size distribution and surface composition.

A “subject” includes any animal, such as a human, monkey, rat, mouse, dog, cat, or fish.

The term “vasculature” as used herein refers to the network of blood vessels in a subject.

### The Contrast Agents

In general, for ease of understanding the present invention, the contrast agents (microspheres, fluorescent dyes, block co-polymer) will first be described in detail, followed by the many and varied methods in which the contrast agents find uses, which is followed by exemplified methods of use.

The utility of an in vivo contrast agent depends on preferential accumulation of the agent in target tissue and achievement of sufficient signal-to-noise ratios to yield satisfactory image resolution. Herein is provided a novel exogenous contrast effector formulation designed to non-invasively detect and monitor various disease states. This colloid-based contrast agent formulation described is designed to be optically excited in tissue, generating fluorescent light useful for collection and formation of images in a living subject. After injection into a subject, the contrast agent can migrate to sites within the body distant from the injection point, and can accumulate in tissues in which excessive or otherwise abnormal blood vessel development occurs as part of disease processes such as inflammation (rheumatoid arthritis), tumor growth (e.g. cancer), fibrodysplasia, various ocular diseases (e.g. diabetic retinopathy), epidermal cell proliferation (psoriasis), brain edema, infertility, cardiovascular pathologies, and bacterial infections related to wound healing.

After treatment with appropriate surfactants(s), the fluorescent microspheres travel relatively freely within the circulating blood until their preferential sequestration occurs at diseased or injured tissue sites, allowing non-invasive imaging of the sites using near-infrared (NIR), or in some cases visible light, for excitation. The formulation described provides an improved means, compared with already-existing optical-based contrast agent formulations, to achieve sensitive and highly localized detection of diseased sites and to image those sites within the body non-invasively.

In a particular aspect, the present microsphere-based contrast agent contains highly size-uniform emulsion-polymerized polystyrene microspheres that comprise a fluorescent dye incorporated within the microsphere. A wide variety of different microspheres may be utilized in the present invention. Preferably, the microspheres (which are small spheres stained with a fluorescent dye having an excitation spectra of about 580 nm to about 800 nm), are composed of biocompatible synthetic polymers or copolymers prepared from monomers such as, but not limited to, acrylic acid, methacrylic acid, ethyleneimine, crotonic acid, acrylamide, ethyl acrylate, methyl methacrylate, 2-hydroxyethyl methacrylate (HEMA), lactic acid, glycolic acid, ε-caprolactone, acrolein, cyanoacrylate, bisphenol A, epichlorhydrin, hydroxyalkylacrylates, siloxane, dimethylsiloxane, ethylene oxide, ethylene glycol, hydroxyalkyl-methacrylates, N-substituted acrylamides, N-substituted methacrylamides, N-vinyl-2-pyrrolidone, 2,4-pentadiene-1-ol, vinyl acetate, acrylonitrile, styrene, p-amino-styrene, p-amino-benzyl-styrene, sodium styrene sulfonate, sodium 2-sulfoxyethylmethacrylate, vinyl pyridine, aminoethyl methacrylates, 2-methacryloyloxy-trimethylammonium chloride, and polyvinylidene, as well polyfunctional crosslinking monomers such as N,N′-methylenebisacrylamide, ethylene glycol dimethacrylates, 2,2′-(p-phenylenedioxy)-diethyl dimethacrylate, divinylbenzene, triallylamine and methylenebis-(4-phenyl-isocyanate), including combinations thereof. In one aspect the polymers include polyacrylic acid, polyethyleneimine, polymethacrylic acid, polymethylmethacrylate, polysiloxane, polystyrene, polydimethylsiloxane, polylactic acid, poly(ε-caprolactone), epoxy resin, poly(ethylene oxide), poly(ethylene glycol), and polyamide (nylon). In another aspect the copolymers include the following: polyvinylidene-polyacrylonitrile, polyvinylidene-polyacrylonitrile-polymethylmethacrylate, and polystyrene-polyacrylonitrile. A most preferred polymer is polystyrene.

The term biocompatible, as used herein in conjunction with the terms monomer or polymer, is employed in its conventional sense, that is, to denote polymers that do not substantially interact with the tissues, fluids and other components of the body in an adverse fashion in the particular application of interest, such as the aforementioned monomers and polymers.

Examples of select microspheres known in the art include, but are not limited to, poly(D,L-lactide-co-glycolide) (PLGA) microspheres; poly(epsilon-caprolactone) (PCL) microspheres; poly(D,L-lactide)/poly(D, L-lactide-co-glycolide) composite microparticles; alginate-poly-L-lysine alginate (APA) microcapsules; alginate microspheres; poly(D,L-lactic-co-glycolic acid) microspheres; chitosan microspheres; poly[p-(carboxyethylformamido)-benzoic an hydride] (PCEFB) microspheres; Hyaluronan-based microspheres; biodegradable microspheres; microspheres of PMMA-PCL-cholesterol; poly(propylene fumarate)/poly(lactic-co-glycolic acid) blend microspheres; poly(lactide-co-glycolide acid-glucose) microspheres; polylactide co-glycolide (PLG) microspheres; poly(methacrylic acid) microspheres; poly(methylidene malonate 2.1.2)-based microspheres; ammonio methacrylate copolymer microspheres; poly(ethylene oxide)-modified poly(beta-amino ester) microspheres; methoxy poly(ethylene glycol)/poly(epsilon-caprolactone) microspheres; polyferrocenylsilane microspheres; poly(fumaric-co-sebacic acid) (P(FASA)) microspheres; poly(lactide-co-glycolide) and poly(ethylene oxide)-poly(propylene oxide)-poly(ethylene oxide) triblock copolymer microspheres; polyisobutylcyanoacrylate microspheres; polystyrene core-glycopolymer corona nanospheres; poly(ethyl-2-cyanoacrylate) microspheres; POE-PEG-POE triblock copolymeric microspheres; poly(DL-lactide) microparticles; albumin microspheres; poly(EGDMA/HEMA) based microbeads; glutaraldehyde crosslinked sodium alginate microbeads; pectin microspheres; methoxy poly(ethylene glycol) and glycolide copolymer microspheres; crosslinked polyethyleneimine microspheres; poly(glycidyl methacrylate-co-ethylene dimethacrylate); cellulose acetate trimellitate ethylcellulose blend microspheres; poly(ester) microspheres; polyacrylamide microcarriers; polyacrolein microspheres; 2-hydroxyethyl methacrylate microspheres

The microspheres may be of varying size. Suitable size microspheres include those ranging from between about 10 and about 5000 nm in outside diameter, preferably between about 50 and about 500 nm in outside diameter. Most preferably, the microspheres are about 75 nm to about 200 nm in outside diameter.

The microspheres of the invention may be prepared by various processes, as will be readily apparent to those skilled in the art, such as by interfacial polymerization, phase separation and coacervation, multiorifice centrifugal preparation, and solvent evaporation, or a combination thereof. Suitable procedures which may be employed or modified in accordance with the present disclosure to prepare microspheres within the scope of the invention include those procedures disclosed in U.S. Pat. Nos. 4,179,546; 3,945,956; 4,108,806; 3,293,114; 3,401,475; 3,479,811; 3,488,714; 3,615,972; 4,549,892; 4,540,629; 4,421,562; 4,420,442; 4,898,734; 4,822,534; 3,732,172; 3,594,326; 3,015,128; Deasy, Microencapsulation and Related Drug Processes, Vol. 20, Chs. 9 and 10, pp. 195-240 (Marcel Dekker, Inc., N.Y., 1984), Chang et al., Canadian J. of Physiology and Pharmacology, Vol 44, pp. 115-129 (1966), and Chang, Science, Vol.146, pp. 524-525 (1964).

Using controlled emulsion polymerization followed by fluorescent dye impregnation, microspheres with a wide range of sizes, surfaces and optical properties can be produced. Although a relatively mature technology in terms of manufacturing and handling, the use of latex or polystyrene microspheres for formulating in vivo contrast imaging agents, and in particular using microspheres impregnated with NIR fluorescent dyes for this purpose, to the best of our knowledge has never been used before.

Any fluorescent dye known to one of skill in the art having an excitation wavelength compatible with in vivo imaging can be used to stain the present microspheres. Typically the fluorescent dyes will have an excitation wavelength of at least 580 nm. A wide variety of long wavelength fluorescent dyes that may be suitable for impregnation in the microspheres are already known in the art (RICHARD P. HAUGLAND, MOLECULAR PROBES HANDBOOK OF FLUORESCENT PROBES AND RESEARCH PRODUCTS (2002)) (Supra).

A fluorescent dye or fluorophore of the present invention is any chemical moiety that exhibits an absorption maximum beyond 580 nm and that is optically excited and observable in tissue. Dyes of the present invention include, without limitation; a pyrene, an anthracene, a naphthalene, an acridine, a stilbene, an indole or benzindole, an oxazole or benzoxazole, a thiazole or benzothiazole, a 4-amino-7-nitrobenz-2-oxa-1, 3-diazole (NBD), a carbocyanine (including any corresponding compounds in U.S. Ser. Nos. 09/968,401; 09/969,853 and 11/150,596 and U.S. Pat. Nos. 6,403,807; 6,348,599; 5,486,616; 5,268,486; 5,569,587; 5,569,766; 5,627,027; 6,664,047; 6,048,982 AND 6,641,798), a carbostyryl, a porphyrin, a salicylate, an anthranilate, an azulene, a perylene, a pyridine, a quinoline, a borapolyazaindacene (including any corresponding compounds disclosed in U.S. Pat. Nos. 4,774,339; 5,187,288; 5,248,782; 5,274,113; and 5,433,896), a xanthene (including any corresponding compounds disclosed in U.S. Pat. No. 6,162,931; 6,130,101; 6,229,055; 6,339,392; 5,451,343 and U.S. Ser. No. 09/922,333), an oxazine or a benzoxazine, a carbazine (including any corresponding compounds disclosed in U.S. Pat. No. 4,810,636), a phenalenone, a coumarin (including an corresponding compounds disclosed in U.S. Pat. Nos. 5,696,157; 5,459,276; 5,501,980 and 5,830,912), a benzofuran (including an corresponding compounds disclosed in U.S. Pat. Nos. 4,603,209 and 4,849,362) and benzphenalenone (including any corresponding compounds disclosed in U.S. Pat. No. 4,812,409) and derivatives thereof. As used herein, oxazines include resorufins (including any corresponding compounds disclosed in U.S. Pat. No. 5,242,805), aminooxazinones, diaminooxazines, and their benzo-substituted analogs. Where the dye is a xanthene, the dye is optionally a fluorescein, a rhodol (including any corresponding compounds disclosed in U.S. Pat. Nos. 5,227,487 and 5,442,045), a rosamine or a rhodamine (including any corresponding compounds in U.S. Pat. Nos. 5,798,276; 5,846,737; 5,847,162; 6,017,712; 6,025,505; 6,080,852; 6,716,979; 6,562,632). As used herein, fluorescein includes benzo- or dibenzofluoresceins, seminaphthofluoresceins, or naphthofluoresceins. Similarly, as used herein rhodol includes seminaphthorhodafluors (including any corresponding compounds disclosed in U.S. Pat. No. 4,945,171). Fluorinated xanthene dyes have been described previously as possessing particularly useful fluorescence properties (Int. Publ. No. WO 97/39064 and U.S. Pat. No. 6,162,931).

Preferred dyes of the invention include xanthene, cyanine, and borapolyazaindacene. Particularly preferred are borapolyazaindacene dyes or dyes sold under the trade name BODIPY.

In one embodiment the dye has an emission spectrum with its maximum greater than about 600 nm. In a further embodiment the dye or fluorophore has an emission spectrum with its maximum greater than about 620 nm, an emission maximum greater than about 650 nm, an emission maximum great than about 700 nm, an emission maximum greater than about 750 nm, or an emission maximum greater than about 800 nm. In particularly preferred embodiment the dye has an emission maximum greater than about 600 nm wherein the microsphere has been impregnated with the dye in a concentration optimized for in vivo imaging. In one aspect the dye is a cyanine dye. Preferred are those dyes sold under the trade name Alexa Fluor® dye or spectrally similar dyes sold under the trade names Cy® dyes, Atto dyes or Dy® dyes. Preferred Alexa Fluor dyes include Alexa Fluor 647 dyes, Alexa Fluor 660 Dye, Alexa Fluor 680 dye, Alexa Fluor 700 dye and Alexa Fluor 750 dye.

Typically the dye contains one or more aromatic or heteroaromatic rings, that are optionally substituted one or more times by a variety of substituents, including without limitation, halogen, nitro, sulfo, cyano, alkyl, perfluoroalkyl, alkoxy, alkenyl, alkynyl, cycloalkyl, arylalkyl, acyl, aryl or heteroaryl ring system, benzo, or other substituents typically present on chromophores or fluorophores known in the art.

In another embodiment, the present microspheres can be impregnated with fluorescent or light scattering nanocrystals [Yguerabide, J. and Yguerabide, EE, 2001 J. Cell Biochem Suppl. 37: 71-81; U.S. Pat. Nos. 6,214,560; 6,586,193 and 6,714,299]. These fluorescent nanocrystals can be semiconductor nanocrystals or doped metal oxide nanocrystals. Nanocrystals typically are comprised of a core comprised of at least one of a Group II-VI semiconductor material (of which ZnS, and CdSe are illustrative examples), or a Group III-V semiconductor material (of which GaAs is an illustrative example), a Group IV semiconductor material, or a combination thereof. The core can be passivated with a semiconductor overlayering (“shell”) uniformly deposited thereon. For example, a Group II-VI semiconductor core may be passivated with a Group II-VI semiconductor shell (e.g., a ZnS or CdSe core may be passivated with a shell comprised of YZ wherein Y is Cd or Zn, and Z is S, or Se). Nanocrystals can be soluble in an aqueous-based environment. An attractive feature of semiconductor nanocrystals is that the spectral range of emission can be changed by varying the size of the semiconductor core.

After selection of an appropriate dye with the desired spectral characteristics, typically where the excitation wavelength is at least 580 nm, or a series of dyes when a longer Stokes shift is desired (U.S. Pat. No. 5,573,909 (herein incorporated by reference)), the dyes are incorporated in a polymeric microparticle, using methods well known in the art. As stated above, the polymeric microparticle can be prepared from a variety of polymerizable monomers, including styrenes, acrylates and unsaturated chlorides, esters, acetates, amides and alcohols, including, but not limited to nitrocellulose, polystyrene (including high density polystyrene latexes such as brominated polystyrene), polymethylmethacrylate and other polyacrylic acids, polyacrylonitrile, polyacrylamide, polyacrolein, polydimethylsiloxane, polybutadiene, polyisoprene, polyurethane, polyvinylacetate, polyvinylchloride, polyvinylpyridine, polyvinylbenzylchloride, polyvinyltoluene, polyvinylidene chloride, and polydivinylbenzene.

In one embodiment of the invention, the present contrast agents are prepared from undyed microparticles. The microparticles can be manufactured in a variety of useful sizes and shapes. They may be spherical or irregular in shape, and range in size from about 0.01 micrometers to about 50 micrometers. Typically, the labeled microparticles are less than about 15 micrometers in diameter and are spherical. More typically the microparticle is a microsphere less than about 5 micrometers in diameter. The microparticles may be of uniform size and/or shape or non-uniform. Alternatively, one or more dyes are added to pre-dyed microparticles such as the many varieties of fluorescent microspheres available commercially, provided that the dyes have an excitation and emission spectra compatible with in vivo imaging. The preferred embodiment has a highly uniform size distribution.

The fluorescent dyes are incorporated into the microparticles by any of the methods known in the art, such as copolymerization of a monomer and a dye-containing comonomer or addition of a suitable dye derivative in a suitable organic solvent to an aqueous suspension of polymer microparticles. For example, the fluorescent microparticles can be produced by free radical-initiated, anaerobic copolymerization of an aqueous suspension of a mono-unsaturated monomer that may or may not contain a covalent bonding group such as carboxyl, amino or hydroxyl and a fluorescent monomer mixture containing at least 10% by weight of monomers comprising the appropriate dye, as defined above. The fluorescent microparticles can also be produced by gradual addition of a solution of the appropriate fluorescent dyes in an appropriate solvent to a stirred aqueous suspension of microparticles, as described by Bangs (UNIFORM LATEX PARTICLES (1984, Seragen, Inc.).

Oil-soluble fluorescent dyes, being freely soluble in organic solvents and very sparingly soluble in water, can easily be introduced by solvent-based addition of the dye to previously manufactured polymer microparticles. This offers the great advantage of being able to prepare uniform polymer microparticles with desired properties by carefully optimized procedures and then later adding the fluorescent dyes of choice. Furthermore, the solvent-based addition process gives great flexibility in adjusting the relative concentrations of the dyes, a key parameter in attaining sufficiently bright fluorescent contrast agents.

In this manner, a large batch of microparticles with desired physical properties, such as size and charge density, can be prepared. Then various fluorescent dyes can be added to smaller portions of this batch resulting in subbatches of fluorescent polymer microparticles with desired spectral properties that give consistent and reproducible performance in applications. In the case of fluorescent microparticles prepared by solvent-based addition of the dye to previously manufactured polymer microparticles, the surfaces properties of the subject fluorescent microparticles are not substantially different from the surface properties of the corresponding undyed microparticles. The fluorescent label in the microparticles is also not affected by changes in pH of the medium surrounding the microparticles.

Furthermore, the dyes used in the subject microparticles are not significantly removed from the microparticles by the water-based solvents that are commonly used as a suspension medium for the microparticles. In addition, the dyes are not “leaked” from the microparticles when in the body or biological fluids. This is an important aspect of the present contrast agents and overcomes many of the known problems with the currently available contrast agents. Another important aspect of the present contrast agents is their ability to localize to sites of injury or disease within the body. The present contrast agent formulation allows for this with the use of a block copolymer on the microspheres providing for adequate circulation of the contrast agents resulting in availability for localization to the appropriate tissue sites.

In one embodiment, a single type of dye is present in the microsphere. In another embodiment, multiple dyes are present in the microsphere. In one aspect the dyes are a series of dyes functioning as an acceptor and donor resulting in a longer Stokes shift than with an individual dye. In another aspect multiple dyes are present that do not have spectral overlap. In this instance, the contrast agent formulation comprises these microspheres in injectable formulations in which two emission bands, for example, one in the visible and one in the NIR range, would be of use. Such applications could include ex vivo post-dissection histology. The NIR emission of the particles would be used to locate the disease feature using a macroscopic imaging system. The diseased tissue with entrapped polymer microspheres could then be more closely examined using microscopes equipped to collect and process visible light.

There exists a body of published scientific literature investigating the in vivo fate of injected microspheres (and many other materials as well) (Gref et al, Science 18: 1600-1603 (1994)). One very consistent feature of these studies is the high potential for injected materials to be rapidly filtered out of the blood by the reticuloendotheial system so that they become sequestered in the liver, spleen, kidneys, or bladder. As the utility of an in vivo contrast agent depends on preferential accumulation of the agent in the tissue of interest, rapid and efficient removal of injected microspheres from the circulation can seriously diminish the likelihood that useful images can be acquired.

Thus, in a preferred embodiment, the present microspheres are coated with a block copolymer to allow the contrast agents to circulate in the body and to accumulate at the site of injury or disease. Without wishing to be bound to a theory, it appears that the use of the block copolymer, or surfactant, facilitates successful imaging by reducing the rate at which organ sequestration of the injected particulates occurs, thus allowing more of the agent to be in contact with diseased sites for a longer time.

The rapid sequestration of intravenously injected particles from the blood by hepatic midzonal and periportal Kupffer cells is problematic for efficient delivery of these types of contrast agents to sites in non-hepatic tissues. It is recognized that clearance behavior and tissue distribution are greatly influenced by the size and surface characteristics of injected particles. Therefore, there has been growing interest in engineering of particulate systems that upon injection avoid rapid recognition by Kupffer cells and adequately remain in the blood. The degree of particle self-association in the blood, as well as opsonization processes, can affect the functional size of particles in vivo, which can effectively increase the size of initially small particles so that they are rapidly removed from the circulation by simple filtration in capillary beds (e.g. in the mouse lung following tail vein injection). The opsonization process is the adsorption of protein entities capable of interacting with specific plasma membrane receptors on monocytes and tissue marcrophages, thus promoting particle recognition and entrapment by these and other immune system cells. Evasion of particle binding to, or uptake by, macrophages is therefore a goal at least partially achievable by interfering with protein adsorption and associated prevention of complement activation. In general, neutral or minimally-charged hydrophilic particles are not efficiently coated with opsonizing complement proteins and as a result tend to be poorly recognized by Kupffer cells.

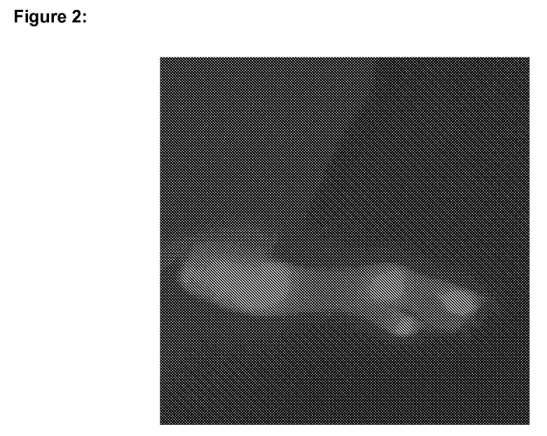

While the tight cell-cell junctions and smooth luminal surface disallow entrapment of the present contrast agents in normal blood vessels, we hypothesize that entrapment of the block copolymer coated fluorescent microspheres occurs in diseased vessels due to their multiple abnormalities, where the particles have a relatively high probability of becoming trapped in rough areas, holes and pits. Another possibility is that at least some part of the in vivo imaging signal derives from microspheres that are engulfed by macrophages that infiltrate and continuously populate diseased tissue areas. See FIG. 2, an image taken using the CRI Maestro Imaging System 24 hr after injection of the disclosed contrast reagent formulation into a female balb/c mouse suffering from experimentally induced arthritis.

In this instance, the formulation tested contains microspheres of approximately 100 nm diameter stained with a BODIPY NIR dye. Microspheres up to 2 microns in diameter have been similarly formulated and tested in an experimental arthritis model in living mice. The general effect is that the larger the diameter, the more rapid and total the clearance of the particle by the reticuloendothelial system, although in the presence of Pluronic F127 copolymer there are enough circulating 2 micron particles to allow imaging of inflamed areas of the mouse fore- and hind-paws after enough time has elapsed for accumulation of material at the inflamed sites (˜48 hr). See FIG. 1.

The effect of surfactants on circulating time of microspheres has previously been documented in the published scientific literature (Moghimi et al. Pharm Rev. 53: 283-318 (2001)), and in particular, Pluronic F127 has been shown effective in this capacity. To the best of our knowledge, the use of surfactants with the present contrast agent has not been used before as part of a contrast agent formulation for in vivo imaging.

Thus in one aspect, the present formulation employs Pluronic F127, a highly water-soluble low toxicity bifunctional block copolymer surfactant present at 2% (w/v) in the contrast agent formulation to reduce organ sequestration of signaling microspheres.

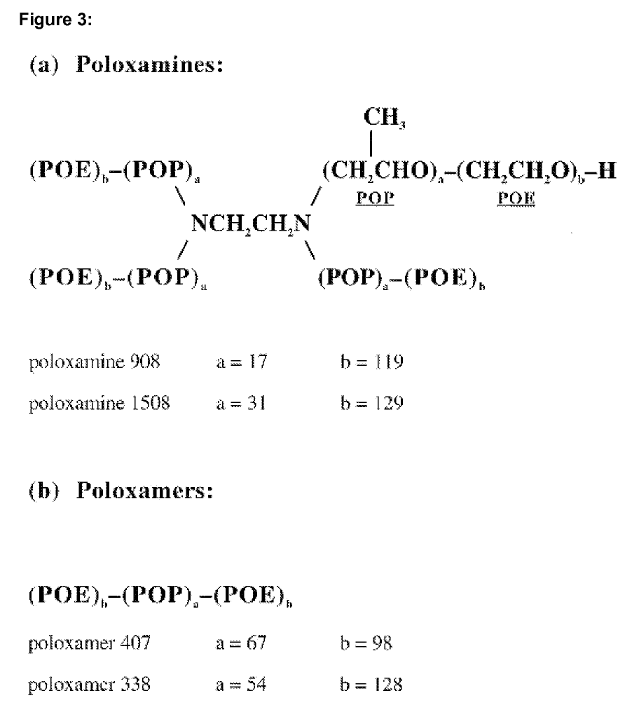

The present polymeric microspheres can be treated with various block copolymers, including ethylene oxide (POE) and propylene oxide (POP) to evade rapid blood clearance (Moghimi, 1997; Stolnik et al, 2001) See FIG. 3. Pre-treatment of microspheres with certain copolymers can improve the circulating blood:liver ratio of injected 60 nm microspheres by up to 10-fold. Numerous investigations have demonstrated that such copolymers adsorb onto the surface of hydrophobic nanoparticulates (e.g. polystyrene, gold, polyphosphazene, poly methyl methacrylate etc.) via their hydrophobic POP center block. This mode of absorption leaves the hydrophilic POE side-arms able to extend outward from the particle surface to provide stability to the particle suspension by a repulsion effect through a steric mechanism of stabilization involving both enthalpic and entropic contributions (Moghimi et al, 1993).

To coat polystyrene microsphere surfaces in the working contrast agent formulation of the instant invention, poloxamers block copolymers of ethylene oxide and propylene oxide can be used, generally having a molecular weight within the range of 1000 to 16,000, and of the structure:

HO(C₂H₄O)_{b}(C₃H₆O)ₐ(C₂H₄O)_{b}H

wherein b is from 2 to 150, and a is from 15 to 70. Generally speaking, block copolymers of ethylene oxide and propylene oxide meeting the above descriptions are available from BASF sold under the trademark “Pluronic and Lutrol F Block Copolymers”. For specifics of such polymers in detail, see BASF Corporation Technical Data Sheets on Pluronic polyols, copyright 1992.

Using the poloxamer coding labels of BASF, suitable poloxamers for use in the invention include, but are not limited to:
Pluronic/Lutrol F 44 (poloxamer 124)
Pluronic/Lutrol F 68 (poloxamer 188)
Pluronic/Lutrol F 87 (poloxamer 237)
Pluronic/Lutrol F 108 (poloxamer 338)
Pluronic/Lutrol F 127 (poloxamer 407)

Polyoxamine tetrafunctional block copolymers, comprising four POE/POP blocks joined together by a central ethylenediamine bridge (See FIG. 3), can also be employed to stabilize and protect the polystyrene surfaces in a closely analogous manner, the goal generally being to produce neutral or minimally-charged hydrophilic particles that are poorly recognized by Kupffer cells in the liver.

It has also been shown that surface modifications with poloxamers and poloxamines before intravenous injection is not always strictly necessary for making nanoparticles long-circulatory. Intravenously injected uncoated 60 nm polystyrene nanoparticles (which are susceptible to phagocytosis by Kupffer cells) were converted to long-circulating entities in rats that received a bolus intravenous dose of either poloxamer-407 or poloxamine-908, 1 to 3 h earlier (Moghimi, 1997, 1999.) It can be argued that the altered biodistribution profile of nanoparticles is the result of cell-surface modification by the administered copolymers. For instance, block copolymers could adhere to cell membrane hydrophobic domains via their hydrophobic center block or act as an effective membrane-spanning entity (Watrous-Peltier et al., 1992). The extracellular steric constraints resulting from hydrophilic POE tails of copolymers will then prevent the interaction between an approaching particle and the cell. Interestingly, this is apparently not the primary mechanism. Instead, nanoparticles have been shown to acquire a coating of copolymer and/or copolymer-protein complexes in the blood (Moghimi, 1997); this event explains their phagocytic resistance.

Microsphere surfaces in the present invention can also be coated with linear polyethylene glycol (PEG) to evade rapid blood clearance. The polymer backbone is essentially chemically inert, and the terminal primary hydroxyl groups are available for derivatization. Usually, the hydroxyl groups are first activated and then reacted with the chosen surface group; PEG activation and functionalization methods have been exhaustively reviewed (Zalipsky, 1995; Monfardini and Veronese, 1998). Surface modification of nanoparticles with PEG and its derivatives can be performed by adsorption, incorporation during the production of nanoparticles, or by covalent attachment to the surface of particles. Examples of currently available PEG conjugates for nanoparticle surface engineering includes PEG-R type copolymers, where R is PLA (Stolnik et al., 1994; Bazile et al., 1995), PLGA (Gref et al., 1994), and poly-ε-caprolactone (Shin et al., 1998; Kim et al., 1998) with appropriate molecular weights. The molecular weight of the PEG segment varies between 2000 and 5000, which is necessary to suppress protein adsorption.

Thus, the present contrast agents are polymeric microspheres formulated to circulate within the body after injection (although inhalation may be another effective route of administration), and to be observable within the unperturbed living animal using optical equipment capable of exciting NIR dyes in the microspheres and of collecting, displaying, and analyzing the fluorescent light emitted from the particles. Controlled variables considered in preparing the imaging formulation include the absolute size and size distribution of the polystyrene particles, degree of chemical crosslinking of constituent polymer chains, optical properties of the dye or dyes used for impregnation, degree of dye loading, surface properties of the particles, state of aggregation of the native dyed particles, and types and amount of additional components (buffers, salts, surfactants, copolymers, etc.) present in the colloidal microsphere particle suspension comprising the injectable contrast imaging agent.

Therefore, in a preferred embodiment, an exemplary formulation to be used with a mouse inflammation disease model, comprises the following parameters:
Particle size: ˜110 nm
Particle size distribution: ±4% diameter
Chemical crosslinking: none
Optical properties of NIR dye: BODIPY dye with excitation max: 715 nm/emission max: 755 nm
Degree of dye loading: maximum attainable
Surface properties: native sulfate
Surface charge starting material: 6 μEq/gram
Aggregation state: non-settling colloidal suspension at 4° C. and 25° C. in complete formulation
suspension fluid: sterile deionized water
Other components: 2% (w/v) Pluronic F-127 copolymer

The present contrast agents can be used in any method known in the art for optical contrast agents wherein the contrast agents preferentially accumulate at the site of injury or disease in tissue. Among the applications for passively accumulating optical contrast agents are sentinel lympth node tracing, endoscopic and colonoscopic or cytoscopic procedures, and cancer detection. In addition to the obvious case of examining skin lesions, colonoscopy, bronchoscopy, upper gastrointestinal endoscopy, and laparoscopy, which all provide surface illumination of deeper epithelial tissue at risk for neoplasia. In humans, fluorescence colonoscopy as well as fluorescent evaluation of other tissues such as bladder, larynx, esophagus and lung have been performed. Rat and mouse colonoscopy or cytoscopy have also been reported in which one may visually inspect tissues in full color (white light illumination) while observing in a NIR channel another independent parameter, such as vascular leakiness or protease activity. In another application, the contrast agents can be used to guide tissue resection during surgical removal of tumors by providing optical contrast between diseased and non-diseased tissue.

After the present fluorescent microspheres have accumulated at the site of disease or injury in the body the contrast agents are visualized using optical imaging instrumentation. A number of optical imaging approaches are known to those skilled in the art, including, but not limited to the method taught in U.S. Pat. No. 5,422,730. These techniques rely on fluorescence, absorption, reflectance, or bioluminescence as the source of contrast, while imaging systems can be based on diffuse optical tomography, surface-weighted imaging (reflectance diffuse tomography), phase-array detection, confocal imaging, multiphoton imaging, or microscopic imaging with intravital microscopy. With the exception of near-infrared fluorescence imaging and superficial confocal and two-photon imaging, these techniques currently are primarily limited to experimental imaging in small animals.

Near-infrared fluorescence imaging relies on light with a defined bandwidth as a source of photons that encounter a fluorescent molecule (optical contrast agent), which emits a signal with different spectral characteristics that can be resolved with an emission filter and captured with a high-sensitivity charge-coupled-device camera.

Fluorescence-based optical imaging instrumentation can be based on planar continuous wave reflectance or time-domain-based phenomena. Time-domain based optical imaging can quantitatively recover depth, volume, concentration, and fluorescent lifetime of different light emitting molecular probes using both photon temporal distribution and intensity data. Imaging instrumentation based on diffuse reflectance is available from various instrument makers, including Cambridge Research and Instrumentation, Inc. (Woburn, Mass.), and VisEn Medical (Woburn, Mass.). Time-domain-based imaging instrumentation is available from GE Healthcare Technologies (Waukesha, Wis.).

### EXAMPLES

The examples below are given so as to illustrate the practice of this invention. They are not intended to limit or define the entire scope of this invention.

### Example 1

### Preparation of 0.1 μm Microspheres with NIR Emission (715 ex/755 em) (Contrast Agent 1)

The staining solution was prepared by adding 700 μL of the dye stock (BODIPY® 670/735 [difluoro(1-((3-(2-(5-hexyl)thienyl)-2H-isoindol-1-yl)methylene)-3-(2-(5-hexyl)thienyl)-1H-isoindolato-N¹,N²)boron] 10 mg/mL stock solution in CH₂Cl₂) to a 10 mL glass test tube, adding 300 μL of CH₂Cl₂, and mixing. 2 mL of Ethanol was then added and sonicated for 30 second to ensure complete mixing. The microspheres were loaded with dye by first adding 10 ml of a vortexed microsphere stock (0.11 μm sulfate polystyrene microspheres (0.1 μm), 8.1% solids, with surface charge content of 6 μEq/g (measured from conductometric titration)) to a 250 ml round bottom flask nad then slowly adding 14 mL of methanol and stirring for 5 minutes. The staining solution was added dropwise to the stirred microsphere suspension and incubated for 30 minutes with continual stirring. The organic solvents were evaporated in a BUCHI R-124 vacuum evaporator, with a water bath setting at 25° C. to prevent possible freeze inside the flask. The stained microspheres were spun for 30 minutes in a centrifuge. The supernatant suspension was then passed through a funnel with a plug of glass wool, into storage bottle.

The excitation and emission spectra were measured and the percentage of solid beads determined. The beads were then coated with a 10% solution of Pluronic F-127 (Invitrogen Corp., P6866) and autoclave with deionized water to make the final microsphere suspension at 1% of solids in 2% of Pluronic F-127. The fluorescent Pluronic F-127 coated microspheres were stored at 4° C.

### Example 2

### Preparation of 2 μm Microspheres with NIR Emission (715 ex/755 em) (Contrast Agent 2)

These fluorescent microspheres were prepared essentially as in Example 1, except that 1.1 mL of dye stock was added to the 10 ml tube and the microsphere stock was 2.0 μm sulfate polystyrene microspheres (8.1% solids, with surface charge content with surface charge content of 6 μEq/g (measured from conductometric titration)).

### Example 3

### Effect of Block Copolymer on Contrast Agent Localization

An effective contrast agent must fulfill two criteria; it must target to point of interest (blood vessels, inflammation or wound) and must stay in circulation rather than being sequestered. Many commercially available agents quickly sequester in the liver when injected systemically leaving only a small fraction of the injected agent in circulation. If the agent is quickly sequestered, it may not have time to reach the target of interest or may quickly leave the target, limiting the time available for imaging. Sequestration is a problem in in vivo imaging because lower concentrations of the material reach the point of interest and the large signal from the liver can overwhelm the signal coming from a rarer target of interest.

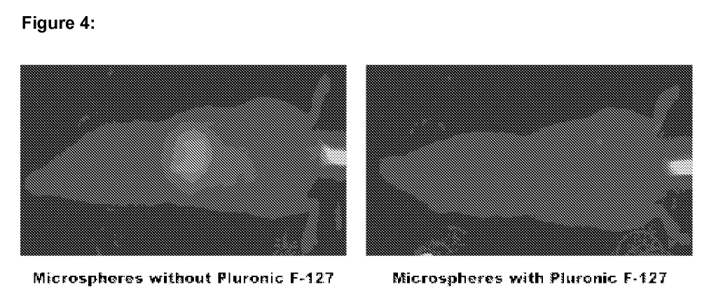

To overcome the problem of sequestration, the block copolymer Pluronic F-127 was added to the microspheres prior to systemic (IV) injection. A direct comparison was made between 2.0 μm polystyrene microspheres embedded with BODIPY 715/755 dye with and without the block copolymer in terms of liver sequestration. The control mouse was injected IV with 100 ul of microspheres (4% solid) in deionized water. The test animal was injected IV with 100 ul of microspheres (4% solid)+2% Pluronic F-127 in deionized water. It was assumed that the injection efficiency was the same in both mice. The mice were imaged ventral side up with a 687 nm excitation/740-950 nm longpass emission filter set and a 500 ms exposure time. See FIG. 4.

### Example 4

### Induction of Arthritis in Mice

Inflammation was modeled by inducing polyarticular collagen-induced arthritis (CIA) in 4-6 week old female Balb/c mice (Charles River). Antibody mediated CIA was induced by intravenous (IV; tail vein) injection of 2 mg Arthrogen-CIA Monoclonal Antibody Blend (Chemicon). Three days after antibody treatment, each mouse received 50 μg Lipopolysaccharide (LPS; Chemicon) intraperitoneally (IP). Seven days after the initial injection, the mice had recovered from the LPS toxicity and symptoms of arthritis were observed. Mice were monitored daily and scored for paw and joint swelling based on the following scale:
0: Normal
1: Mild but definite swelling of ankle or wrist or swelling limited to individual digits
2: Moderate redness and swelling of ankle and wrist
3: Severe redness and swelling of the entire paw including digits (none of the study mice reached this stage)

Mice were injected with the present contrast agent when they reached level 2, between five and ten days after the LPS injection.

Inflammation persisted for 14-21 days; after this time, the swelling decreased visibly. Bone degradation and ankylosis were not observed, though according to the product literature provided by Chemicon may have continued until day 28.

Twenty percent of the induced mice did not develop arthritis; this failure rate was expected based on the product literature. Mice that did not develop inflammation within 28 days were not used in imaging experiments.

### Example 5

### Determination of Testing Parameters

### Dose Determination

The optimal dosage of contrast agent was determined using a simple wound healing assay. We serendipitously developed an assay for blood flow and pooling in healing tissue after noting that microspheres injected systemically pooled in the margins of tissue around ear punches. The mouse's ear was punched at the time of agent injection simply as an identification mark but the ear tissue labeling timecourse closely mimicked that of inflamed tissue in arthritic animals. The ear punch model used less expensive, normal animals and did not require complicated injections or a lag time while the animals developed disease. Normal Balb/c mice were injected intravenously (IV; tailvein) with 100 μl of 0.1 μm microspheres at concentrations of 2%, 1%, 0.5% or 0.25% solids in water. The ear was immediately marked using a manual ear punch. The labeling was imaged 24 hours post-injection using the Ex 687/Em 740-950 filter set and a 100 ms exposure.

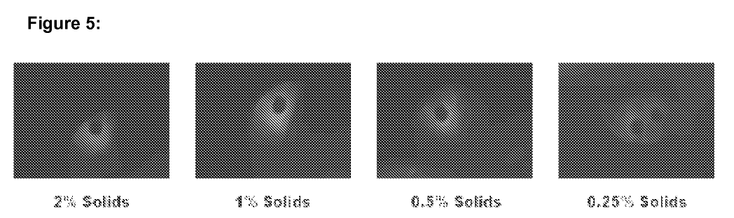

The 0.5% and 0.25% concentrations were eliminated due to the weak signal. The 2% concentration was eliminated due to the signal bleed; even with shorter exposure times, the labeling appeared strong yet diffuse throughout the ear rather than labeling the healing areas specifically. The final concentration of 1% solids was chosen based on the strong binding to the new vessels around the ear punch and lack of signal bleed. See FIG. 5.

### Injection of Reagent

Contrast agents were introduced systemically by IV injection. A 29-gauge needle was used to inject 100 μl of 1% solids in water (without buffer salts or saline) containing 2% Pluronic F-127 via the lateral tail vein. The tail was stroked lengthwise three times with an alcohol saturated pad to dilate the blood vessels prior to injection.

### Toxicity of Agent

No overt acute toxicity was observed in any of the Balb/c mice used to test the P10S or P200S contrast agents at the recommended dose (or in preliminary studies carried out with C57/BLK6 mice). No evidence of capillary occlusion was observed. All of the animals were still alive and healthy (based on feeding, drinking, excretion, posture and behaviour) at the conclusion of the study. All of the study animals continued to be monitored by OVSAC personnel for sixty days to rule out the possibility of long-term toxicity.

### Imaging Conditions

Mice were anesthetized by inhalation of 2.5% isoflurane/oxygen in a tabletop induction chamber equipped with a warming pad. During imaging, the mice were maintained at 2% isoflurane in the imaging chamber. The mice were allowed to recover in fresh air between timepoints spaced more than 30 minutes apart.

The Maestro™ 500 In-Vivo Imaging System (Cambridge Research & Instrumentation, Inc) was used to acquire multispectral image files. The system uses a 300 watt Xenon light source and tunable emission filters. The system acquires cubes of images spaced 10 nm apart through the emission spectral range which can be spectrally unmixed to allow differentiation of targets based on their emission profiles, or to subtract out autofluorescence to allow detection of low intensity signals. Images can also be saved as RGB composites which can more easily be used for qualitative analysis.

In a typical experiment, the mouse was placed ventral side up in the imaging chamber with the stage at position 1 to image the mouse on a whole animal level. A standard exposure of 500 ms at each emission wavelength was used to ensure that the data from each timepoint could be directly compared. A cube of images at 10 nm intervals required 9-20 seconds to acquire (depending on the emission filter set used). The full set of scans required three minutes due to the time required to switch filter sets. The following excitation/emission conditions were used:
Ex 529 nm/Em 570-950 nm (CRI: Green Filter Set)
Ex 640 nm/Em 690-950 nm (CRI: Red Filter Set)
Ex 687 nm/Em 740-950 nm (CRI: Deep Red Filter Set)
Ex 735 nm/Em 790-950 nm (CRI: NIR Filter Set)

Each paw of the animal was also imaged individually to provide better signal resolution. The stage platform was moved to position 3 for maximal magnification. The paws were imaged with a standard 100 ms exposure time with an excitation wavelength of 687 nm and emission from 740-950 nm at 10 nm increments.

Images of the mice treated with Contrast Agent 1 were obtained at 10 min, 30 min, 90 min, 3 hours, 4.5 hours, 6 hours, 24 hours, 48 hours, 3 days, 7 days, 14 days, 21 days and 28 days post-injection.

Images of the mice treated with Contrast Agent 2 were obtained at 30 min, 90 min, 2.5 hours, 24 hours, 48 hours, 7 days and 14 and 21 days post-injection.

A Balb/c mouse which had been injected with Arthrogen-CIA Monoclonal Antibody Blend and Lipopolysaccharide (LPS; Chemicon) but did not develop detectable arthritis was used as a control. The mouse was imaged at the individual paw and full body levels with all four filter combinations to provide a control spectral library for use in image analysis.

### Example 6

### Qualitative Analysis of Targeting, Contrast Agent 1

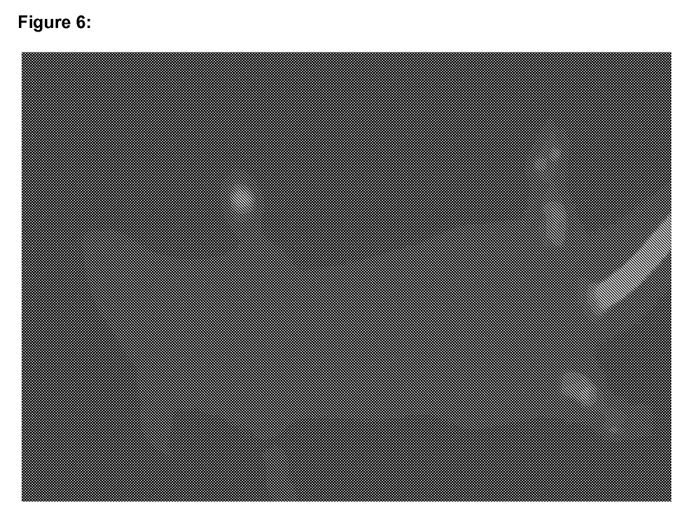

The mouse used to test Contrast Agent 1 displayed arthritic swelling in three paws (front right, rear right and rear left). The visually asymptomatic paw (front left) served as an internal control. The contrast agent clearly labeled the three affected paws, but not the normal paw. The agent was specific to the sites of inflammation; there was little accumulation observed (on the basis of optical signal) in the liver, spleen or intestine over the twenty eight day time course. See FIG. 6.

To more clearly demonstrate the signal time course, a selection of images from a single paw is shown below. The inflamed areas were faintly visible two hours after injection and were clearly visible after 24 hours. The signal strength gradually decreased over the twenty eight day imaging period, but the localized signal was clearly visible for the entire time course.

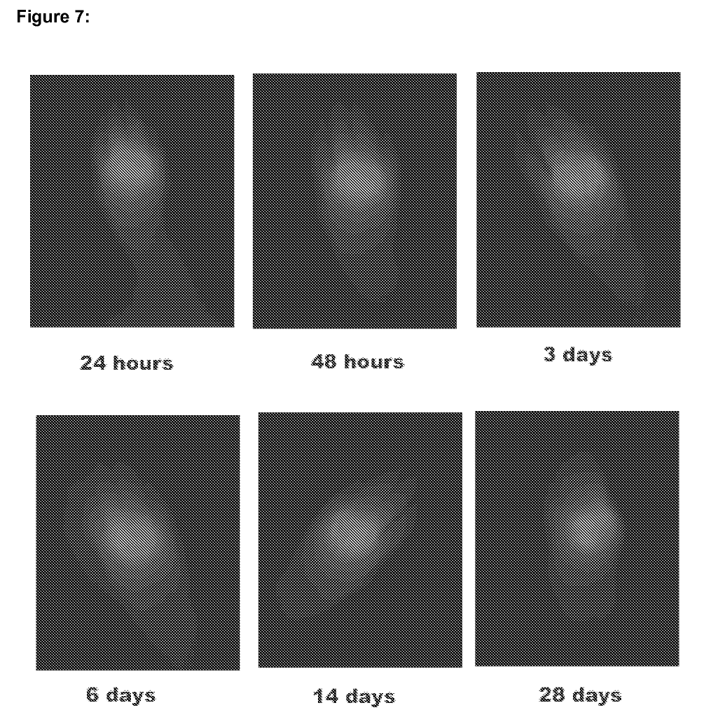

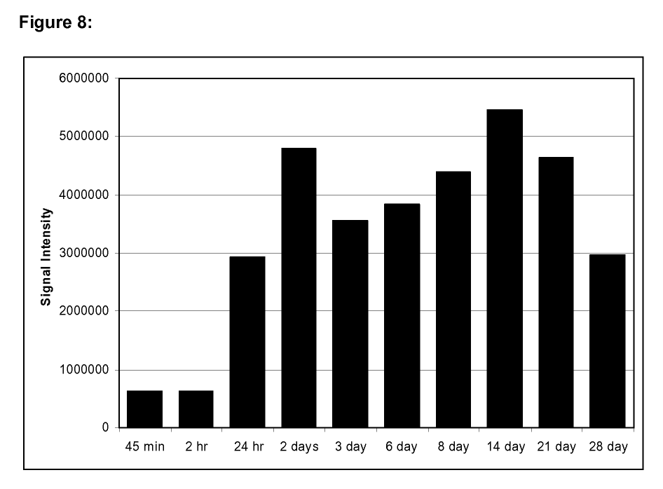

The Contrast Agent 1 provided the good resolution and allowed resolution of the individual knuckles and joints, whereas the BSA conjugate labeled the whole paw. This excellent resolution was maintained for 28 days post-injection, while the contrast agent 2 greatly decreased in resolution after fourteen days. See FIGS. 7 and 8.

### Example 7

### Qualitative Analysis of Targeting, Contrast Agent 2

The mouse used to test Contrast Agent 2 did not have as severe of inflammation. At the whole mouse level, it appeared that only the heel joints were affected. However, when higher magnification was used to image the individual paws, the inflammation in individual joints of the front right paw was clearly visible.

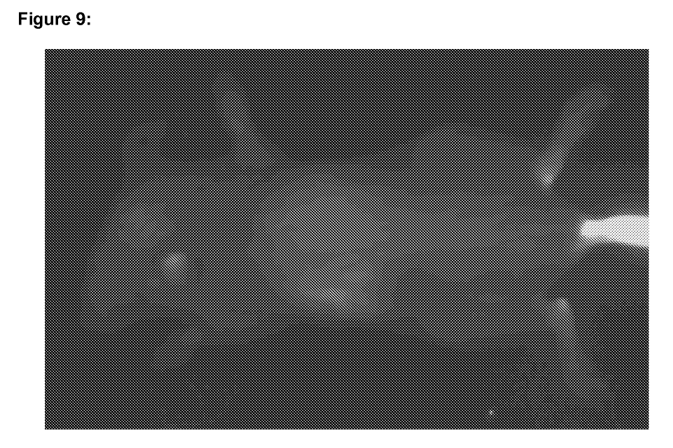

Contrast agent 2 was not as specific as Contrast agent 1. A portion of the material was observed sequestered in the liver within ninety minutes of the injection. This signal remained visible in the liver throughout the time course. See FIG. 9. The time course of targeting to sites of inflammation in individual joints of the front right paw is shown below. The joints were faintly labeled at 30 minutes post-injection, but the highest resolution was seen after 24 hours. At this point, three inflamed joints were clearly visible. The labeling was still distinct seven days after injection; after fourteen days, the signal strength, along with the resolution had notably decreased.

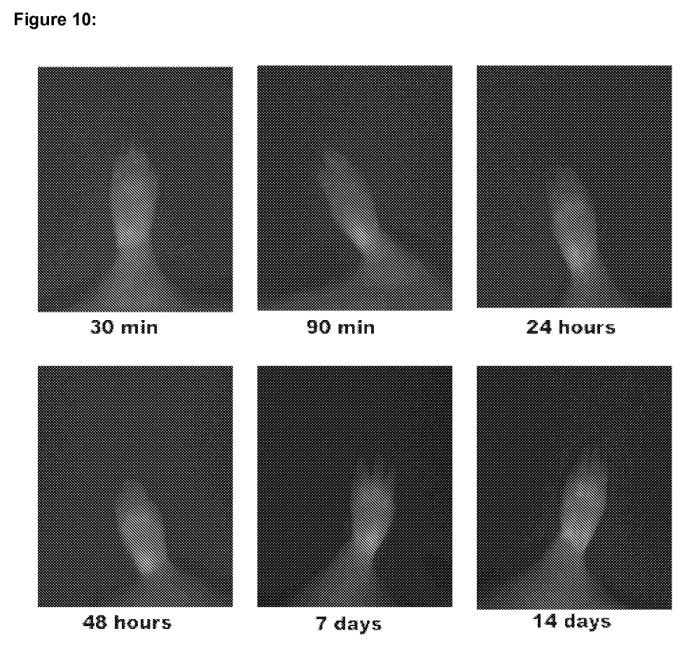

The Contrast Agent 2 labeled inflamed areas with resolution at the level of the individual knuckles and joints, but this high resolution labeling did not last as long as the Contrast agent 1 labeling; contrast agent 2 greatly decreased in resolution after fourteen days while high resolution was maintained for 28 days post-injection using contrast agent 1. The labeling was however superior to transferrin and BSA in terms of resolution and circulation time. See FIG. 10.

### Example 8

### Preparation of Sterile Microspheres (Contrast Agent 1)

The stained microspheres of Example 1 were brought to a concentration of 1% solids using 0.2 micron filtered autoclaved water freshly delivered from a deionized source. The bead suspension (about 80 mL total) was transferred to a 100 mL PYREX brand media bottle, graduated, with a plug-seal cap. The suspension was then Autoclaved in IDC's Sterilemax table top steam sterilizer (Barnstead Thermolyne, Dubuque, Iowa), using “liquid cycle”, 15 minutes at 121° C. Alternatively, the Mediquip Eagle 2000 sterilizer in Packaging Department is used for autoclaving (liquid cycle, 15 minutes at 121° C.). After removal from the autoclave, the solution was cooled to room temperature and transferred to storage in a laminar flow hood at 4° C. as needed.

Where a pellicle (very thin film) was observed on the surface of the cooled autoclaved bead suspension (film may cover ⅓ or more of the surface area), a sterile individually wrapped 25 mL pipet (w/o cotton plug) was used to remove the film by spooling it onto the side of the flat end of the inverted pipette.

After removal of the pellicle, the suspension was warmed to room temperature and centrifuged at 2,200 rpm (˜x 1,000 g) for 15 min at room temperature. Samples were observed with and without pellets. If no pellets were observed the bead suspension was transferred to a sterile media bottle with screw-on cap and mixed well by gentle swirling. Where dark blue pellets were observed on the bottom of the tubes after centrifugation, the suspension was carefully transferred to a media bottle so the pellet of precipitates was not disturbed or any part of the sedimented material transferred.

Detergent was added and the bead suspension adjusted to 0.5% solids with sterile 10% Pluronic F-127 solution and 2% Pluronic F-127 (Described in Example 1). The Pluronic F-127 solutions used in the dilution of beads suspension were 0.2 μm sterile-filtered and handled with sterile technique. Using a sterile pipette, the combined suspension was removed for testing. The emission intensity consistently increases more than 2-fold upon autoclaving of samples.

### Example 9

### Post-Sterilization 0.1 Micron Dosage in Ear Punch Model

The optimal dosage of sterilized contrast agent was determined using the wound mouse ear punch assay described in Example 5. Normal Balb/c mice were injected intravenously (IV; tail vein) with 100 μl of 0.1 μm microspheres in 2% Pluronic F-127 at concentrations of 0.9%, 0.3% or 0.1% solids, sterilized according to Example 8. The ear was immediately marked using a manual ear punch. The labeling was imaged 24 hours post-injection and four days post-injection using the Ex 640/Em 690-950 filter set and a 200 ms exposure.

Results: The 0.1% concentration resulted in a weak signal. The 0.9% solids concentration gave a very strong signal, but showed some signal bleed. The final concentration of 0.3% solids was optimal based on the strong binding to the new vessels around the ear punch and lack of signal bleed.

### Example 10

### Post-Sterilization 0.1 Micron Arthritis Testing

The specificity and time course of sterilized microsphere targeting was determined using the arthritis model described in Example 4. The mouse used to test the autoclaved sterilized contrast agent displayed arthritic swelling in three paws (front right, rear right and rear left). The visually asymptomatic paw (front left) served as an internal control. The contrast agent clearly labeled the three affected paws, but not the normal paw. The agent was specific to the sites of inflammation; there was no visible accumulation (on the basis of optical signal) in the liver, spleen or intestine over the three day timecourse.

The targeting specificity of the autoclaved microspheres is very similar to that of the optimal dosage of non-sterilized product. The autoclave treatment does not affect the ability of the product to circulate through the bloodstream and accumulate at points of inflammation.

### Example 11

### Microsphere Specifications (Heat-Sterilized 2 Micron Microsphere Formulation)

Concentration approximately 1% latex solids from in-process determination
Unit Size 1.05 mL
Medium deionized water with 2% total (w/v) Pluronic F-127 (from MPI P6866)

### Data on Raw Microspheres Starting Material:

| | |
|---|---|
| Particle Size | 2 ± 0.2 μm measured by TEM |
| Surface | Sulfate |
| Charge Content | 2.5 ± 1 μEq/g measured by potentiometric titration |
| Surfactant | Surfactant-free microsphere starting material |

### Fluorescence:

| | |
|---|---|
| Excitation Maximum | 715 ± 10 nm |
| Emission Maximum | 755 ± 10 nm |

### Microscopy:

Few or no aggregates after sonication

### Flow Cytometry:

| | |
|---|---|
| Percentage of Singlets | ≧85% |
| Fluorescence in FL3 | 750 ± 100 |
| (channel values) | Median Intensity |

All publications referred to within this document are incorporated by reference to the same extent as if each individual publication was specifically and individually indicated to be incorporated by reference.

The reagents employed in the examples are commercially available or can be prepared using commercially available instrumentation, methods, or reagents known in the art. The foregoing examples illustrate various aspects of the invention and practice of the methods of the invention. The examples are not intended to provide an exhaustive description of the many different embodiments of the invention. Thus, although the forgoing invention has been described in some detail by way of illustration and example for purposes of clarity of understanding, those of ordinary skill in the art will realize readily that many changes and modifications can be made thereto without departing from the spirit or scope of the appended claims.

We claim:

1. A method for imaging a site of disease or injury within the vasculature of a subject wherein the method comprises:
administering to the subject a contrast agent comprising fluorescent microspheres labeled with a dye having an excitation and emission spectrum compatible with in vivo imaging and wherein the microspheres are coated with a surfactant;
incubating said subject for a sufficient amount of time for the contrast agents to circulate to the disease or injury sites;
illuminating the contrast agents in the subject with an appropriate wavelength to form an illuminated subject; and
observing the illuminated subject whereby the site of disease or injury is imaged.

2. The method of claim 1, wherein the surfactant is a block copolymer.

3. The method of claim 2, wherein the block copolymer is comprised of polyoxyethylene (PEO) and polyoxypropylene (PPO) subunits.

4. The method of claim 3, wherein the block copolymer is poloxamer 407.

5. The method of claim 1, wherein the microspheres comprise polystyrene.

6. The method of claim 1, wherein the dye has an excitation wavelength between about 580 nm to about 800 nm.

7. The method of claim 1, wherein the disease is arthritis, a coronary infarction, an infection, or cancer.

8. The method of claim 1, wherein the dye is selected from the group consisting of a pyrene, an anthracene, a naphthalene, an acridine, a stilbene, an indole, an oxazole, benzoxazole, a thiazole, a benzothiazole, a 4-amino-7-nitrobenz-2-oxa-1,3-diazole (NBD), a carbocyanine, a carbostyryl, a porphyrin, a salicylate, an anthranilate, an azulene, a perylene, a pyridine, a quinoline, a xanthene, an oxazine, a benzoxazine, a carbazine, a phenalenone, a coumarin, a benzofuran, a benzphenalenone, a semiconductor nanocrystal, and a fluorescent protein.

9. The method of claim 1, wherein the contrast agent is administered intravenously to the subject.

10. The method of claim 1, wherein the microspheres are coated with the surfactant in vivo.

11. The method of claim 10, wherein the surfactant is a block copolymer.

12. The method of claim 1, wherein the contrast agent remains at the disease or injury site for at least one hour.

13. The method of claim 1, wherein the contrast agent remains at the disease or injury site for at least five hours.

14. A contrast agent for in-vivo imaging of disease or injury sites in a subject comprising a fluorescent microsphere, wherein the microsphere is labeled with a dye having an excitation and emission spectrum compatible with in vivo imaging and wherein the microspheres are coated with a surfactant.

15. The contrast agent of claim 14, wherein the surfactant is a block copolymer.

16. The contrast agent of claim 15, wherein the block copolymer is comprised of polyoxyethylene (PEO) and polyoxypropylene (PPO) subunits.

17. The contrast agent of claim 16, wherein the block copolymer is poloxamer 407.

18. The contrast agent of claim 14, wherein the microspheres comprise polystyrene.

19. The contrast agent of claim 14, wherein the dye has an excitation wavelength between about 580 nm to about 800 nm.

20. The contrast agent of claim 14, wherein the subject is suffering from a disease selected from arthritis, a coronary infarction, an infection, or cancer.

21. The contrast agent of claim 14, wherein the dye is selected from the group consisting of a pyrene, an anthracene, a naphthalene, an acridine, a stilbene, an indole, an oxazole, benzoxazole, a thiazole, a benzothiazole, a 4-amino-7-nitrobenz-2-oxa-1,3-diazole (NBD), a carbocyanine, a carbostyryl, a porphyrin, a salicylate, an anthranilate, an azulene, a perylene, a pyridine, a quinoline, a xanthene, an oxazine, a benzoxazine, a carbazine, a phenalenone, a coumarin, a benzofuran, a benzphenalenone, a semiconductor nanocrystal, and a fluorescent protein.

22. A kit for in-vivo imaging of disease or injury sites in a subject comprising:
a contrast agent comprising a fluorescent microsphere, wherein the microsphere is labeled with a dye having an excitation and emission spectrum compatible with in vivo imaging and wherein the microspheres are coated with a surfactant;
packaging; and
instructions for imaging the disease or injury site with the contrast agent.

23. The kit of claim 22, wherein the surfactant is a block copolymer.

24. The kit of claim 23, wherein the block copolymer is comprised of polyoxyethylene (PEO) and polyoxypropylene (PPO) subunits.

25. The kit of claim 24, wherein the block copolymer is poloxamer 407.

26. The kit of claim 22, wherein the microspheres comprise polystyrene.

27. The kit of claim 22, wherein the dye has an excitation wavelength between about 580 nm to about 800 nm.

28. The kit of claim 22, wherein the subject is suffering from a disease selected from arthritis, a coronary infarction, an infection, or cancer.

29. The kit of claim 22, wherein the dye is selected from the group consisting of a pyrene, an anthracene, a naphthalene, an acridine, a stilbene, an indole, an oxazole, benzoxazole, a thiazole, a benzothiazole, a 4-amino-7-nitrobenz-2-oxa-1,3-diazole (NBD), a carbocyanine, a carbostyryl, a porphyrin, a salicylate, an anthranilate, an azulene, a perylene, a pyridine, a quinoline, a xanthene, an oxazine, a benzoxazine, a carbazine, a phenalenone, a coumarin, a benzofuran, a benzphenalenone, a semiconductor nanocrystal, and a fluorescent protein.

30. The kit of claim 22, wherein the contrast agent is for intravenous administration to the subject.

31. The kit of claim 22, wherein the contrast agent is suspended in deionized water.
